(19) United States
(12) Patent Application Publication
(10) Pub. No.: US 2008/0287943 A1
(43) Pub. Date: Nov. 20, 2008

(54) TREATMENT APPARATUS AND METHODS FOR INDUCING MICROBURN PATTERNS IN TISSUE

(75) Inventors: Bryan Weber, Livermore, CA (US); Bader-Eddine Bellahsene, San Mateo, CA (US); Edward Ebbers, San Carlos, CA (US); Mitchell Levinson, Pleasanton, CA (US); Adnan Merchant, Fremont, CA (US); Karl Pope, San Mateo, CA (US)

Correspondence Address: WOOD, HERRON & EVANS, LLP, 2700 CAREW TOWER, 441 VINE STREET, CINCINNATI, OH, 45202, US

(73) Assignee: THERMAGE, INC., Hayward, CA (US)

(21) Appl. No.: 12/019,874

(22) Filed: Jan. 25, 2008

Related U.S. Application Data
(60) Provisional application No. 60/886,587, filed on Jan. 25, 2007.

(51) Int. Cl. A61B 18/18; A61B 18/04; A61F 2/00

(52) U.S. Cl. 606/41; 606/32; 607/101

(57) ABSTRACT

Treatment apparatus and methods for inducing microburn patterns in tissue. The treatment apparatus comprises a delivery device positionable adjacent to the tissue and a plurality of energy-transfer elements. The energy-transfer elements are adapted to contact the skin surface over discrete surface contact areas and transfer energy to the tissue for forming damaged regions in the form of microburns at a corresponding plurality of locations in the tissue. Energy may be transferred between the energy-transfer elements and the tissue by either electrical conduction or thermal conduction. Adjacent microburns are separated by non-damaged regions, which promotes wound healing and efficacy.

### CROSS-REFERENCE TO RELATED APPLICATIONS

This application claims the benefit of U.S. Provisional Application No. 60/886,587, filed Jan. 25, 2007, which is hereby incorporated by reference herein in its entirety.

### FIELD OF THE INVENTION

The invention generally relates to apparatus and methods for treating tissue and, more particularly, relates to apparatus and methods for inducing microburn patterns in tissue.

### BACKGROUND OF THE INVENTION

Lasers have been utilized in conjunction with cosmetic surgical techniques for delivering a pattern of discrete microscopic thermal wounds or microburns to the skin and underlying tissue. The use of lasers to form microburns is disclosed in for example, U.S. Published Application Nos. 2006/0206103; 2006/0009668; 2005/0049582; 2004/0082940; 2003/0216719, and U.S. Pat. No. 6,997,923; the disclosure of each of which is hereby incorporated by reference herein in its entirety. The depth of the microburns is limited such that tissue damage is not caused below a predetermined depth of the skin surface. The microburns are likewise locally confined in an area such that the temperature rise of the tissue between adjacent microburns is minimized. The cells in the regions of undamaged tissue spared between adjacent microburns operate as seeds for the regrowth of rejuvenated tissue, which replaces the tissue damaged by the microburns as the wounds heal.

Lasers and other optical sources may be disadvantageous because their depth of tissue penetration is relatively shallow. The shallow penetration depth limits the depth of the tissue that can be treated. In particular, essentially no subcutaneous tissue can be treated with laser and optical sources. When the therapeutic effect being desired is tissue tightening and tissue contouring in a Z direction perpendicular to the skin surface, these effects cannot be optimally realized with such sources because of the limited tissue penetration.

What is needed, therefore, are apparatus and methods capable of delivering thermal energy to epidermis, dermis, or subcutaneous tissue to form a microburn pattern.

### SUMMARY OF THE INVENTION

Embodiments of the invention are generally directed to treatment apparatus and methods that are configured to induce microburn patterns in tissue located beneath a skin surface.

In accordance with an embodiment of the invention, a device is provided for forming a plurality of damaged regions in tissue interspaced between non-damaged or undamaged regions. Both the damage and undamaged regions are located beneath a skin surface. The device comprises an electrode assembly positionable adjacent to the skin surface, the electrode assembly comprising a plurality of energy-delivery elements, the energy-delivery elements configured to deliver energy to the tissue for forming the damaged regions at a corresponding plurality of locations in the tissue, which are separated by another plurality of undamaged tissue regions.

In another embodiment of the invention, the device may comprise a delivery device positionable adjacent to the skin surface. The delivery device comprises a plurality of thermally-conductive elements configured to contact the skin surface. The device includes a fluid delivery member configured to deliver a coolant to the thermally-conductive elements for cooling the thermally-conductive elements to a temperature sufficient to thermally form damaged regions at a corresponding plurality of locations in the tissue separated by another plurality of non-damaged or undamaged tissue regions.

In another embodiment of the invention, a method is provided for forming a plurality of damaged regions characteristic of a microburn pattern in tissue beneath a skin surface. The method comprises transferring high frequency electrical energy between a plurality of small-area tissue contacts and the tissue and modifying the tissue with the transferred energy to form the tissue damaged regions correlated with the small-area tissue contacts such that the damaged regions are separated by a plurality of tissue non-damaged regions.

In another embodiment of the invention, a method is provided for forming a plurality of damaged regions characteristic of a microburn pattern in tissue beneath a skin surface. The method comprises transferring heat energy from the tissue over a plurality of small-area tissue contacts. The method further comprises cooling the tissue at the small-area tissue contacts with the heat energy transfer to an extent sufficient to form the damaged regions in the tissue correlated with the small-area tissue contacts such that the damaged regions are separated by a plurality of non-damaged regions in the tissue.

In certain embodiments of the invention, the conductively transferred energy may be in a band of the electromagnetic spectrum outside of the band in the electromagnetic spectrum characteristic of laser operation. Embodiments of the invention may also rely on contact between a portion of the delivery device and the skin surface for conductive energy transfer, which differs from the intrinsically non-contact laser methods used conventionally for forming microburns. The contact for the conductive energy transfer may be either direct or indirect through a conductive coupling fluid.

### BRIEF DESCRIPTION OF THE DRAWINGS

The accompanying drawings, which are incorporated in and constitute a part of this specification, illustrate embodiments of the invention and, together with a general description of the invention given above and the detailed description of the embodiments given below, serve to explain the principles of the invention.

FIG. 1 is a perspective view of a delivery device for thermally delivering a microburn pattern to tissue in accordance with an embodiment of the invention.

FIG. 2 is a side view of the delivery device of FIG. 1.

FIG. 3 is a cross-sectional view of a delivery device for thermally delivering a microburn pattern to tissue in accordance with an alternative embodiment of the invention.

FIG. 4 is a cross-sectional view of a delivery device for thermally delivering a microburn pattern to tissue in accordance with an alternative embodiment of the invention.

FIG. 5 is a diagrammatic view of a delivery device for thermally delivering a microburn pattern to tissue in accordance with an alternative embodiment of the invention.

FIG. 6 is a top view in partial cross-section of a delivery device for thermally delivering a microburn pattern to tissue in accordance with an alternative embodiment of the invention.

FIG. 7 is a side view in partial cross-section of a delivery device for thermally delivering a microburn pattern to tissue in accordance with an alternative embodiment of the invention and in which a treatment tip is lifted from the skin surface.

FIG. 8 is a cross-sectional side view similar to FIG. 7 in which the treatment tip is contacting the skin surface.

FIG. 9 is a top view of a delivery device for thermally delivering a microburn pattern to tissue in accordance with an alternative embodiment of the invention and in which a dielectric layer supplying electrical insulation for the electrode array has been omitted for clarity of description.

FIG. 9A is a diagrammatic cross-sectional view taken generally along line 9A-9A of FIG. 9 in which the electrodes are operating in a bipolar mode.

FIG. 10 is a perspective view of a handpiece for use with the delivery devices of FIGS. 1-9 in accordance with the invention.

FIG. 11 is a side view in partial cross-section of a delivery device for thermally and cryogenically delivering a microburn pattern to tissue in accordance with an alternative embodiment of the invention.

FIG. 12 is a perspective view of a delivery device for thermally and cryogenically delivering a microburn pattern to tissue in accordance with an embodiment of the invention.

FIG. 13 is a side view in partial cross-section of the delivery device of FIG. 10.

### DETAILED DESCRIPTION

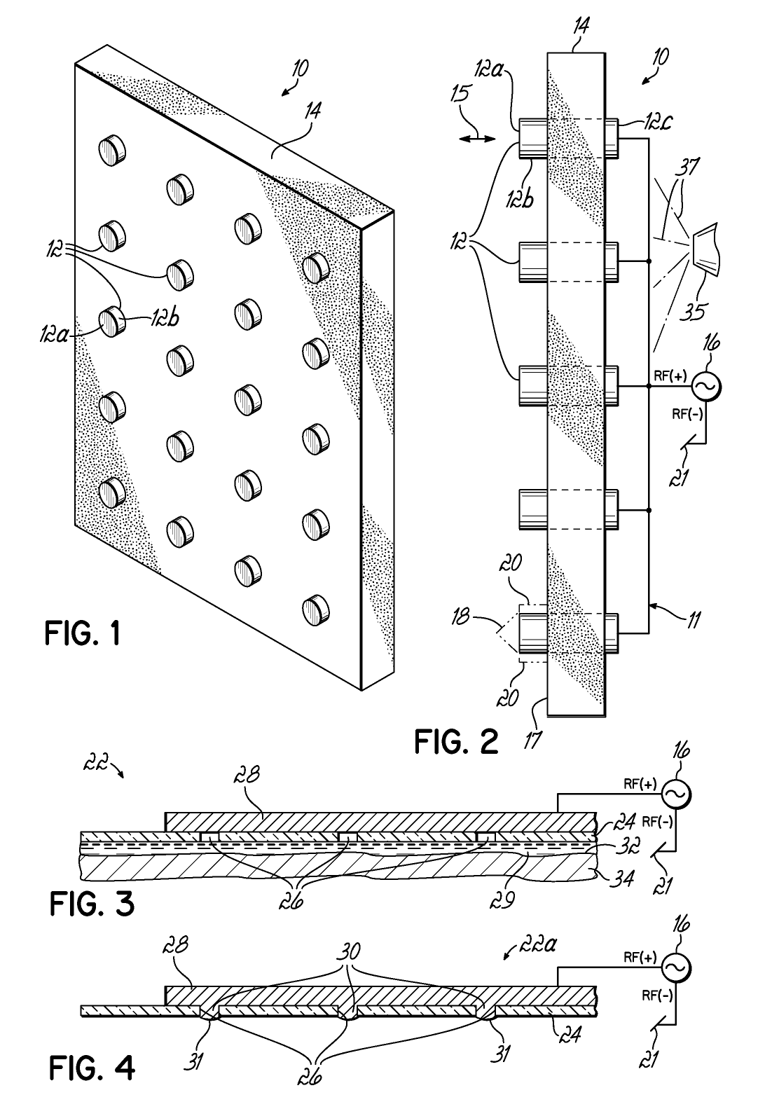

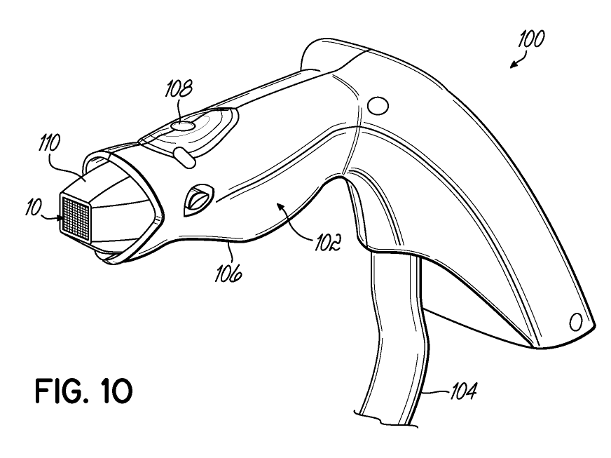

With reference to FIGS. 1 and 2, a delivery device 10 for a treatment apparatus or handpiece 100 (FIG. 10) includes an array of individual electrodes 12 that penetrate through a dielectric member 14. The electrodes 12, which are formed from an electrically-conductive material, are electrically coupled in an electrical circuit, generally indicated by reference numeral 11, with the positive polarity voltage terminal of a high frequency generator or power supply 16. In the representative embodiment, the treatment handpiece 100 is adapted to be grasped by a clinician for placing the electrodes 12 of the delivery device 10 in contact with, or otherwise proximate to, a patient's skin surface 32. Once contacted with, or proximate to, the skin surface 32 (FIG. 3), the array of electrodes 12 radiates high frequency electromagnetic energy into a tissue 34 (FIG. 3) of the patient, which lies beneath the skin surface 32. The tissue 34 may be the dermis, epidermis, or subcutaneous tissue. The delivery device 10 may include additional sensors, such as impedance, pressure or thermal sensors (not shown).

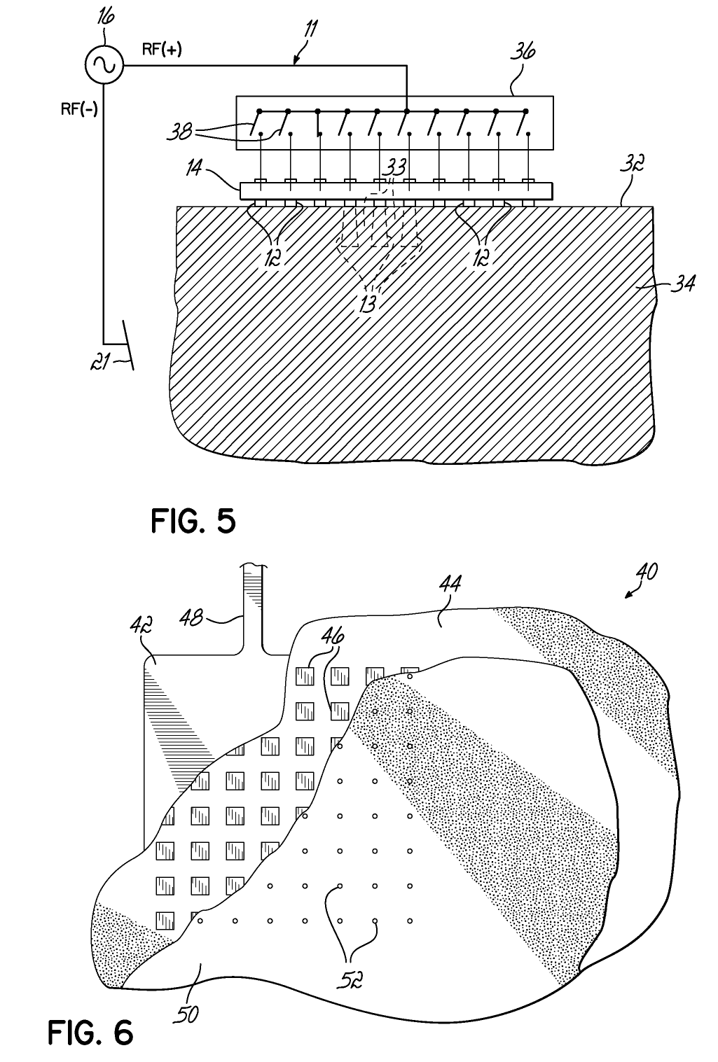

The high frequency power supply 16 is operative to generate high frequency electrical current, typically in the radio-frequency (RF) region of the electromagnetic spectrum, which is transferred to the electrodes 12 in the delivery device 10. The operating frequency of power supply 16 may advantageously be in the range of several hundred KHz to about 40 MHz, preferably about 1 MHz to about 10 MHz, more preferably about 4 MHz to about 8 MHz, to impart a therapeutic effect to the tissue 34 that is effective to create microburns 13 (FIG. 5) in the tissue 34. Optionally, a capacitor or inductor can be placed in the electrical circuit with the power supply 16 as desired for achieving system impedance matching. The power supply 16 converts a line voltage into drive signals having an energy content and duty cycle appropriate for the amount of power and the mode of operation that have been selected by the clinician for the treatment, as understood by a person having ordinary skill in the art.

A non-therapeutic passive or return electrode 21 (FIG. 5) is attached to a body surface of the patient (i.e., the patient's leg or back) removed or remote from a treatment zone (i.e., face, arm, hand, abdomen, etc.) and is electrically coupled with a negative voltage polarity terminal of the high frequency power supply 16. During treatment, high frequency current flows through the bulk of the patient between the delivery device 10 and the return electrode 21. Current delivered by the delivery device 10 is returned to the high frequency power supply 16 from the return electrode 21, after having been conducted through the tissue 34 of the patient, to close the electrical circuit 11. The return electrode 21 is non-therapeutic in that only insignificant heating is produced at its attachment site to the patient's body because of the low current density delivered across the relatively large area of the return electrode 21, which is significantly larger in area than the collective area of all the electrodes 12.

When the electrodes 12 are energized and are placed into contact with the external skin surface 32, high frequency energy delivered to the tissue 34 generates a pattern of discrete microscopic thermal wounds or microburns 13 comprising tissue damaged regions in the target tissue 34. The microburns 13 are generated by tissue heating arising from high-frequency current radiating outwardly and inwardly into the tissue 34 from each electrode 12. Tissue 34 conducts electrical current with some degree of electrical resistance, which creates localized heating of the tissue 34 through which the current is being conducted and, thereby, creates the microburns 13. Depending on the amount of energy delivered, tissue temperatures of about 40° C. (about 104° F.), about 45° C. (about 113° F.), about 50° C. (about 122° F.), about 55° C. (about 131° F.), about 60° C. (about 140° F.), and about 65° C. (about 149° F.) are easily obtainable. To achieve the higher temperatures without creating second or third degree burns, surface skin cooling can be utilized as understood by those skilled in the art. Surface skin cooling is taught in commonly-assigned U.S. Pat. No. 6,350,276, which is hereby incorporated by reference herein in its entirety. The dielectric member 14 has a substantially planar surface 17 that is approximately parallel to the tissue 34 when the microburns 13 are formed and from which the electrodes 12 project toward the skin surface 32.

Typically, cells in the microburns 13 in the tissue 34 die at temperatures greater than about 45° C. (about 113° F.) and the cells disintegrate as the temperature increases beyond cell death temperature. Cell death creates a wound healing response from the body over a period spanning two days to six months following treatment. New collagen is formed during the wound healing, which imparts a tighter and younger appearance to the skin surface 32. In addition and in different embodiments, existing collagen denatures during the treatment when tissue temperature exceeds about 50° C. (about 122° F.), about 55° C. (about 131° F.), or about 60° C. (about 140° F.). Collagen denaturization may result in immediate tissue tightening such that the skin looks (and is) tighter and more youthful looking. Tightening occurs in the x and y directions (i.e., in a plane containing the skin surface 32) and, when collagen is denatured in the subcutaneous tissue 34, tightening in the z direction (i.e., in a normal direction perpendicular to the plane containing the skin surface 32) also occurs which makes the patient look more youthful and younger.

The microburns 13 are formed as damaged regions at a corresponding plurality of locations in the tissue 34. Adjacent locations of the microburns 13 are separated by healthy regions 33 of tissue 34, which may be substantially unaffected (i.e., non-damaged or undamaged) by the treatment process. Typically, the microburns 13 constitute small, closely-spaced, and isolated zones or columns of damaged tissue 34 that are surrounded by regions 33 of healthy tissue 34. When cooling is not employed, the regions 33 of healthy tissue 34 are separated from damaged tissue 34 in the microburns 13. The tissue heating spreads outwardly from the zone in which the energy is delivered to form the microburns 13.

When cooling is employed, it is possible to protect the entire tissue 34 from damage, if desired, by regulating the amount of cooling and energy delivery. In any case, the invention contemplates also creating as desired, damaged dermal tissue 34 separated from undamaged dermal tissue 34, and damaged subcutaneous tissue 34 separated from undamaged subcutaneous tissue 34. The depth and extent of tissue damage can be readily controlled by controlling the amount of energy delivered to the tissue 34, and by controlling the amount of cooling delivered to the tissue 34. In one embodiment, a nozzle member 35, which may include multiple individual nozzles, may be used to deliver a spray 37 of a cryogen or coolant toward the electrodes 12 for controlling the temperature of the electrodes 12 and, thereby, the temperature of the patient's tissue 34.

The close proximity of the healthy regions 33 of epidermal, dermal, and/or subcutaneous tissue 34 surrounding each individual microburn 13 supports rapid healing of the damaged tissue to create new healthy tissue 34 to replace skin imperfections, remodel collagen, and tighten skin texture. In an outpatient procedure, the treatment may be used to erase features such as pigmented areas, acne scars, surgical scars, melasma, and sun spots, and to eradicate fine, medium, and deep wrinkles.

The treatment depth may be adjusted by, for example, programming different output parameters (i.e., high frequency currents and voltages, duration over which current is applied, etc.) for the high frequency power supplied from power supply 16 to the delivery device 10. Cooling can be adjusted by providing a pre-treatment cooling period, a concurrent-treatment cooling period, a post-treatment cooling period, as desired, and also by controlling the temperature of the treatment tip during the cooling to be, for example, either extremely cold, medium cooled, or mildly cooled, as desired. The treatment depth may also be contingent upon other variables, such as the specific type of tissue 34 involved in the treatment.

The delivery device 10 is moved among successive treatment locations for treating large regions, such as the patient's face, with patterns of microburns 13. Multiple passes over the treatment zone separated by a few minutes may be used to enhance the treatment, as is understood by persons skilled in the art. Multiple treatments, which are separated temporally by a healing period, may be needed for a successful treatment that supplies the desired cosmetic effect.

Each electrode 12 has a leading or forward end 12a that defines a small area contact that directly contacts the skin surface 32 during treatment. Each electrode 12 further includes a sidewall 12b that connects the forward end 12a with an opposite rearward end 12c. The rearward end 12c of each electrode 12 is connected in the electrical circuit 11 with the high frequency power supply 16. An optional conductive sheet (not shown) may be provided on the non-patient side of the delivery device 10 to promote efficient electrical connection of the electrodes 12 in the electrical circuit 11 with the high frequency power supply 16. High frequency energy in the form of an electrical signal or current is conducted along the length of each electrode 12 from the rearward end 12c to the forward end 12a.

The sidewall 12b of each electrode 12 projects a short distance beyond the dielectric member 14 such that, during treatment, the forward end 12a may penetrate into the target tissue 34. The length of the exposed portion of each electrode 12 is selected such that the electrodes 12 do not deflect or bend when pressed against the skin surface 32. For example, the exposed length of the electrodes 12 may be approximately equal to the diameter (e.g., 100 μm) of the exposed portion. Alternatively, the exposed length of the electrodes 12 may be on the order of about 2 times to about 10 times the diameter of the exposed portions. In specific embodiments, the exposed length of the electrodes 12 may be on the order of about 2, about 3, about 4, about 5, or about 10 times the diameter of the exposed portion.

The forward end 12a of each of the electrodes 12 may be pressed or pushed against the skin surface 32 with a force sufficient to depress the skin surface 32 and tissue 34 and, thereby, to form a concavity in the skin surface 32 and tissue 34 centered about each individual electrode 12. This may assist in reducing the divergence of the delivered high frequency energy with penetration depth into the tissue 34, which reduces the diameter of the approximately-circular cross-section heated zone. Because of this divergence, the diameter of the microburns 13 increases with increasing penetration depth into the tissue 34. All electrodes 12 may be energized concurrently to impart the pattern of microburns 13 in the tissue, although the invention is not so limited.

In one specific embodiment of the invention, the electrodes 12 in the array are conductive or metallic pins with an exposed length or portion that may be shaped generally as a right circular cylinder. In use, the tips of the pins define small-area tissue contacts with the skin surface 32 for high-frequency energy delivery to the underlying tissue 34. The inter-pin spacing between adjacent pins in the plane of the dielectric member 14 (i.e., the electrode-to-electrode spacing) may range from about 50 μm to about 2000, from about 50 μm to about 3000 μm, or from about 50 μm to about 4000 μm. In specific embodiments, the electrode-to-electrode spacing between adjacent pins in the plane of the dielectric member 14 may be approximately 100 μm, approximately 200 μm, approximately 300 μm, approximately 400 μm, approximately 500 μm, approximately 700 μm, approximately 1000 μm, or even approximately 2000 μm. For example, the delivery device 10 may comprise one hundred individual pins arranged in a 10×10 rectangular array to provide a one (1) cm² tissue treatment area. Other representative array sizes are in a range of about 1.5 cm² to about 20 cm². Particularly useful array sizes may be about 1.5 cm², about 3 cm², about 5 cm², about 7 cm², about 10 cm², about 15 cm², and about 20 cm². The pin width (diameter for round pins) for each electrode 12 may be in a range of about one-half to about one-twentieth of the electrode-to-electrode spacing. Particularly useful pin widths are approximately one-half, approximately one-third, approximately one-fifth, approximately one-tenth, or approximately one-twentieth of the electrode-to-electrode spacing. An exemplary pin width is approximately 100 μm for an electrode-to-electrode spacing of about 1000 μm, and 200 μm for an electrode-to-electrode spacing of about 2000 μm.

The electrodes 12 may be modified to promote penetration beneath the skin surface 32 and into the tissue 34. To that end and in accordance with one embodiment, each electrode 12 may be compliantly coupled with the dielectric member 14. When pressed against the skin surface 32, the forward end 12a of each individual electrode 12 may retract relative to the substantially planar surface 17 of the dielectric member 14. As the transferred high frequency energy modifies the tissue 34, the forward end 12a of each individual electrode 12 may be advanced forward to define the microburns 13 and, thereby, create a path for advancement of the forward end 12a, as indicated diagrammatically by double-headed arrow 15 (FIG. 2). The movement direction of the electrodes 12 is in a direction substantially perpendicular to the substantially planar surface 17 of the dielectric member 14. Alternatively, the forward end 12a of each electrode 12 may be sharpened to define an optional beveled point 18 (FIG. 2) that promotes penetration into tissue 34 with either static electrodes 12 or electrodes 12 capable of retraction/advancement to the dielectric member 14.

In another embodiment, the sidewall 12b of the exposed portion of each electrode 12 may be covered by an optional dielectric shroud 20. The dielectric shroud 20 is formed from a material having a higher electrical resistivity (i.e., lower electrical conductivity) than a material forming the electrodes 12. As a result, only the surface area of the forward end 12a is in direct electrical conduction contact with the tissue 34 to direct most, if not all, of the energy through the surface area of the forward end 12a.

With reference to FIG. 3 and in accordance with an alternative embodiment of the invention, a delivery device 22, which operates in a manner similar to delivery device 10, includes a dielectric member 24 and a solid electrode 28 coupled in a contacting manner with the dielectric member 24. The dielectric member 24 contacts the skin surface 32 during treatment. At various locations, the dielectric member 24 is perforated by a plurality of vias or openings in the form of passageways 26. The passageways 26 may be formed in the dielectric member 24 by, for example, a laser drilling process, etching or similar process known to people skilled in the art.

The dielectric member 24 may comprise a flexible sheet or substrate of material, such as a thin base polymer (e.g., polyimide) film. The solid electrode 28 may comprise a thin conductive (e.g., copper) pad. The material forming the dielectric member 24 has a higher electrical resistivity (i.e., lower electrical conductivity) than the conductive material forming the electrodes 12. The delivery device 22 may comprise a flex circuit having a patterned conductive (i.e., copper) foil comprising the solid electrode 28 laminated to a base polymer (or other non-conductive material) film comprising the dielectric member 24. Alternatively, the delivery device 22 may comprise a patterned conductive (i.e., copper) metallization layers comprising the solid electrode 28 directly deposited on a base polymer film comprising the dielectric member 24 by, for example, a physical vapor deposition technique, such as sputter deposition in a vacuum environment. Flex circuits, which are commonly used for flexible and high-density electronic interconnection applications, have a construction understood by a person having ordinary skill in the art.

Alternatively, the dielectric member 24 of the delivery device 22 may comprise a photoimageable (LPI) coverlayer in which passageways 26 are defined. Suitable LPI coverlayer materials include, but are not limited to, the Pyralux® line of photoimageable coverlayers commercially available from DuPont Electronic Materials (Research Triangle Park, N.C.) or the R/Flex® line of photoimageable covercoats commercially available from Rogers Corporation (Chandler, Ariz.).

In use, the dielectric member 24 of the delivery device 22 is contacted with the skin surface 32 and high frequency energy or current is delivered from the solid electrode 28 through the passageways 26 to the tissue 34. Amounts of a conductive coupling fluid 29 may be applied to the skin and can fill the open space in each opening 26 between the conductor of the solid electrode 28 and the skin surface 32 for providing the requisite current paths having a low coupling impedance with the skin. High frequency current is conducted through the conductive coupling fluid 29 filling each opening 26 for conductive transfer to the skin surface 32 and the underlying tissue 34. The conductive coupling fluid 29 may be, for example, an aqueous solution containing one or more electrolytes (i.e., salts) in a concentration sufficient to provide moderate conductivity.

With reference to FIG. 4 and in accordance with an alternative embodiment of the invention, a delivery device 22a is depicted that is similar to delivery device 22 (FIG. 3). Each of the passageways 26 in the dielectric member 24 is filled by a corresponding one of a plurality of plugs 30 of conductive material that is in electrical continuity with the solid electrode 28. The plugs 30 may comprise an integral portion of the solid electrode 28 formed by, for example, an electroplating process that fills the passageways 26. A forward end or crown 31 of each plug 30 may project beyond the plane defined by the dielectric member 24 for providing a small contact area with the skin surface 32, which eliminates the need for a coupling fluid. Rather than having a proud presentation as depicted in FIG. 4, the plugs 30 may be flush with the plane of the dielectric member 24.

With reference to FIG. 5 and in accordance with an alternative embodiment of the invention, a structure for electrically coupling one of the delivery devices 10 (FIGS. 1 and 2), 22 (FIG. 3), or 22a (FIG. 4) with the high frequency power supply 16 is depicted in conjunction with delivery device 10. The electrodes 12 are electrically coupled in the electrical circuit 11 with the high frequency power supply 16 by a multiplexing switchbox or network 36 that controls the application of high frequency energy to the electrodes 12. The multiplexing network 36 includes a plurality of switches 38, such as a relay or another type of switching device, that may be switched between opened and closed conditions to open and close, respectively, a signal path between the electrodes 12 and the positive voltage polarity terminal of the high frequency power supply 16. The multiplexing network 36 is used to switch the application of power to different groups of electrodes 12 such that only a fraction of the total number of electrodes 12 is energized at any one time during treatment.

In one embodiment of the invention, high frequency power is switched successively to each of the individual electrodes 12 by a corresponding one of the switches 38. For example, a 10×10 array of electrodes 12 would require a bank or group of one hundred switches 38 to address the entire array. In another embodiment of the invention in which the number of individual switches 38 is comparatively reduced, high frequency power may be switched to an entire row or column of electrodes 12 in the electrode array by a corresponding one of the switches 38. For example, a 10×10 array of electrodes 12 would require a bank or set of ten switches 38 each controlling the application of high frequency power to one row or, alternatively, to one column of ten electrodes 12 in the electrode array. Alternatively, the switches 38 may be connected to the electrodes 12 such that non-column, non-row sets of electrodes 12 in the electrode array are selectively addressed and energized by sequentially opening and closing each of the switches 38. For example, every other electrode 12 in alternating rows of the electrode array may be selectively addressed and energized by opening and closing the switches 38.

With reference to FIG. 6 and in accordance with an alternative embodiment of the invention, a delivery device 40 is depicted that has a base construction based upon a flex circuit design. The delivery device 40 includes a sheet electrode 42 of a relatively large surface area, a dielectric member 44 and a plurality of small electrodes 46 of significantly smaller surface area than sheet electrode 42. The dielectric member 44, which is characterized by a higher electrical resistivity (i.e., lower electrical conductivity) than the conductive material forming the electrodes 42, 46, electrically isolates the sheet electrode 42 from the small electrodes 46.

Delivery device 40 operates in a manner similar to delivery devices 10 (FIGS. 1 and 2), 22 (FIG. 3) and 22a (FIG. 4). The sheet electrode 42 of delivery device 40 is electrically coupled with the high frequency power supply 16 (FIG. 5) by a conductive trace 48. The small electrodes 46 are positioned between the dielectric member 44 and a cover layer 50 of a non-conducting or insulating material. The cover layer 50, which is placed in a contacting relationship with the skin surface 32 (FIG. 5) during treatment to form the microburns 13 (FIG. 5), may be an LPI coverlayer

Passageways or openings 52 perforate the cover layer 50 at locations correlated with the locations of the small electrodes 46. Each opening 52 may be, for example, centered over a corresponding one of the small electrodes 46, or may overlap with a side edge of a corresponding one of the small electrodes 46. A surface area of each of the small electrodes 46 that is visible to the skin surface 32 (FIG. 5) through the corresponding opening 52 provides direct electrical conduction to the tissue 34 (FIG. 5). The visible surface area of each small electrode 46 is less than the total surface area because the non-visible surface area is insulated from the skin surface 32 by a portion of the cover layer 50. The openings 52 extending in a direction from the small electrodes 46 to the tissue 34 and each of the openings 52 is registered with a corresponding one of the small electrodes 46.

The flex circuit construction of the delivery device 40 may include a thin base polymer (e.g., polyimide) film that operates as dielectric member 44, a conductive copper lamination foil or metallization layer bonded to one side of the polymer film that operates as sheet electrode 42, and another conductive copper lamination foil or metallization layer bonded to the opposite side of the polymer film that has been patterned to define the small electrodes 46. The cover layer 50 is applied to the dielectric member 44 after the small electrodes 46 are formed. The openings 52 may have a diameter of approximately 0.005 inch (approximately 0.0127 centimeter) to approximately 0.010 inch (0.0254 centimeter).

In use, high frequency power is capacitively coupled from the sheet electrode 42 though the dielectric member 44 to the small electrodes 46. The delivered power is then conducted from the small electrodes 46 as high frequency current confined, when delivered to the skin surface 32, within the cross-sectional area of the openings 52. The dielectric member 44 between the sheet electrode 42 and the small electrodes 46 causes the sheet electrode 42 to participate in forming an electrical capacitor with each of the smaller electrodes 46 and the dielectric member 44 when the electrodes 42, 46 are energized. It is believed that capacitively coupling the high frequency power will operate to improve the uniformity of energy delivery to the tissue 34 for forming the microburns 13. The dielectric material of the cover layer 50 surrounding each opening 52 prevents energy transfer from the non-visible surface area of the corresponding one of the small electrodes 12.

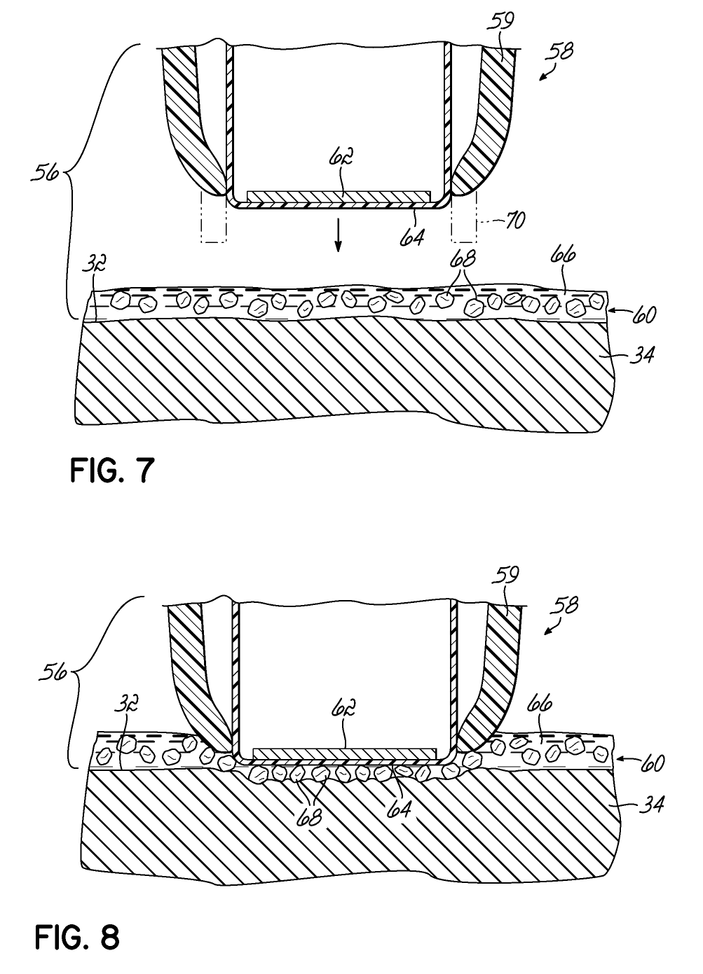

With reference to FIGS. 7 and 8 and in accordance with an alternative embodiment of the invention, a delivery device 56 comprises an electrode assembly, generally indicated by reference numeral 58, and a fluid composition 60 that electrically couples high frequency power from the electrode assembly 58 to tissue 34 for forming microburns 13 (FIG. 5). The electrode assembly 58, which is packaged inside a housing 59, is designed to capacitively couple high frequency energy from a sheet electrode 62, which is coupled with a high frequency power supply 16 (FIG. 5), through the thickness of a dielectric member 64. The material forming the dielectric member 64 has a higher electrical resistivity (i.e., lower electrical conductivity) than the conductive material forming the sheet electrode 62.

The fluid composition 60 is a slurry consisting of a carrier fluid 66 and a plurality of electrically conductive particles 68 that are suspended and carried within the carrier fluid 66. The conductive particles 68 may be spherical or non-spherical and may be formed from any suitable conductive material having a melting point such that conductive particles 68 remain intact during treatment. The viscosity and surface tension of the fluid composition 60 is such that it remains substantially in position on the patient during treatment. Materials for the conductive particles 68 include, but are not limited to, metals such as stainless steels. A person having ordinary skill in the art will appreciate that any particulate material that possesses appropriate conductive properties may be employed in the invention as the conductive particles 68. The carrier fluid 66 may be any of a variety of fluids or semi-fluids with suitable viscosity and surface tension. In an exemplary embodiment, the carrier fluid 66 is non-conducting, or semi-conducting, but not purely conductive.

The conductive particles 68 facilitate selective z-axis coupling of the capacitively-coupled energy from the electrode 62 to the tissue 34. The conductive particles 68 concentrate the electric field supplied by the capacitively-coupled energy and operate to disrupt the uniform, volumetric electric field that would be present in the absence of conductive particles 68. High frequency current conducted by the conductive particles 68 operate to create the microburns 13 (FIG. 5). Among the variables influencing energy transfer are the size of the conductive particles 68, geometry of the electrode assembly 58, and spacing between the electrode assembly 58 and the skin surface 32.

In use, the electrode assembly 58 is moved to capture an amount of the fluid composition 60 between the dielectric member 64 and the skin surface 32. A number of the conductive particles 68 will likewise be captured between the dielectric member 64 and the skin surface 32 at random locations, as shown in FIG. 8. The conductive particles 68 conductively transfer high frequency energy to the tissue 34 for forming the microburns 13 (FIG. 5).

In an alternative embodiment of the invention, the dielectric member 64 may be omitted from the electrode assembly 58. The electrode 62 is separated from the skin surface 32 by a standoff 70 (FIG. 7) arranged about the perimeter of electrode 62. The standoff 70 prevents contact between the electrode 62 and the skin surface 32 during treatment to form microburns 13 (FIG. 5). As an alternative to the standoff 70, the electrode 62 may include a grid of ridges (not shown) that elevates the electrode 62 from the skin surface 32 in a non-contacting relationship. When the electrode 62 is energized, a number of minute conductive paths are created from the electrode 62 through the conductive particles 68 to the tissue 34 at randomized locations. Variables influencing energy transfer include, but are not limited to, the size of the conductive particles 68 and the density and static suspension capability of the carrier fluid 66.

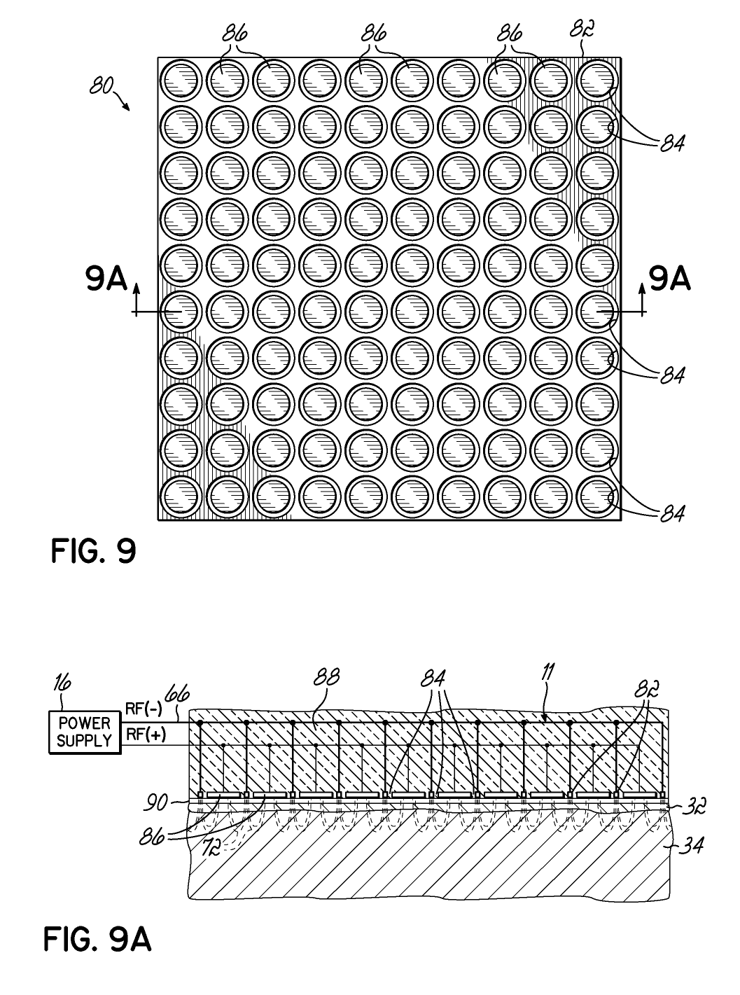

With reference to FIGS. 9 and 9A and in accordance with an alternative embodiment of the invention, a delivery device 80 comprises a conductive sheet electrode 82 including an array of voids 84 and a plurality of secondary electrodes 86 each positioned in a corresponding one of the voids 84. The voids 84 and secondary electrodes 86 are arranged in a matrix of rows and columns within the peripheral boundary of the sheet electrode 82, although the invention is not so limited. The sheet electrode 82 and secondary electrodes 86 are each constituted by an electrically conductive material, such as a metal like copper, gold, silver, aluminum, alloys of these materials, and the like. The sheet electrode 82 and secondary electrodes 86 have a substantially flat planar interleaved structure. The voids 84 and secondary electrodes 86 may be interleaved in a different manner with the sheet electrode 82 so that discrete rows and columns are absent from the arrangement or have a non-rectangular arrangement.

Portions of a dielectric layer define individual insulators 88 electrically insulate the secondary electrodes 86 from the sheet electrode 82. A thin dielectric layer 90, which may be formed from the same dielectric material as the insulators 88, covers a patient facing side of the electrodes 82, 86. When the delivery device 80 is positioned proximate to the skin surface 32, the dielectric layer 90 defines a substantially planar tissue treatment surface that at least partially contacts the skin surface 32. Suitable dielectric materials for insulators 88 and dielectric layer 90 include any ceramic, polymer, or glass having an appropriate dielectric constant and dielectric strength as understood by a person having ordinary skill in the art. The delivery device 80 may be constituted by a multilayer flex circuit, as described herein, such that the electrodes 82, 86 comprise conductive features formed on a surface of a flexible substrate. Alternative fabrication techniques for forming delivery device 80 may include ceramic printed circuit fabrication methods, multilayer rigid printed circuit board fabrication methods, and any other fabrication techniques that involve forming three-dimensional patterns of conductors and dielectrics.

The sheet electrode 82 is electrically coupled with a negative voltage polarity terminal of the high frequency power supply 16. The secondary electrodes 86 are electrically coupled with a positive voltage polarity terminal of the high frequency power supply 16. Consequently, the electrodes 82, 86 operate in a bipolar mode such that the polarity is alternated between any two adjacent electrodes 82, 86 and a return electrode is not required on the patient to complete the current path with the high frequency power supply 16. Instead, the sheet electrode 82 operates to deliver high frequency energy to the tissue 34 for forming the microburns 13 (FIG. 5) and the secondary electrodes 86 supply return current paths 72. The electrical connections between the high frequency power supply 16 and the electrodes 82, 86 may comprise multiple layers or levels of conductive traces or features in which each individual conductive feature layer is electrically isolated from adjacent levels with vias supplying paths between levels or may comprise discrete conductors or wires.

With reference to FIG. 10, a treatment apparatus or handpiece 100 includes a housing 102 with which one of the delivery devices of FIGS. 1-9, such as delivery device 10, is mechanically coupled. Housing 102 typically comprises a plastic material that is molded, such as by an injection molding process, into a three-dimensional shape. A hollow interior of the housing 102 houses electrical connections (not shown) that electrically couple the delivery device 10 in the electrical circuit 11 with the high frequency power supply 16 (FIG. 5). Housing 102 provides a suitable interface for connection to a cable 104 that includes insulated and shielded conductors or wires (not shown) that electrically couple the delivery device 10 with the high frequency power supply 16.

A smoothly contoured grip portion 106 of the handpiece 100 is shaped to be gripped and handled by a clinician for manipulating the handpiece 100 to place the delivery device 10 at a location proximate to a patient's skin surface 32 (FIG. 5). An activation button 108 is depressed and released for controlling the delivery of high frequency energy from the delivery device 10 to the tissue 34 (FIG. 5). The delivery device 10 may be integrated into a removable nozzle tip 110 so that the nozzle tips 110 may be easily interchanged for providing different treatments. The nozzle tips 110 may be disposable after one or more uses. A handpiece suitable for use as treatment handpiece 100 is shown and described in commonly-assigned U.S. Application No. 60/728,339, entitled “Treatment Apparatus Having Multiple Selectable Depths of Energy Delivery” and filed on Oct. 19, 2005; the disclosure of which is hereby incorporated by reference herein in its entirety.

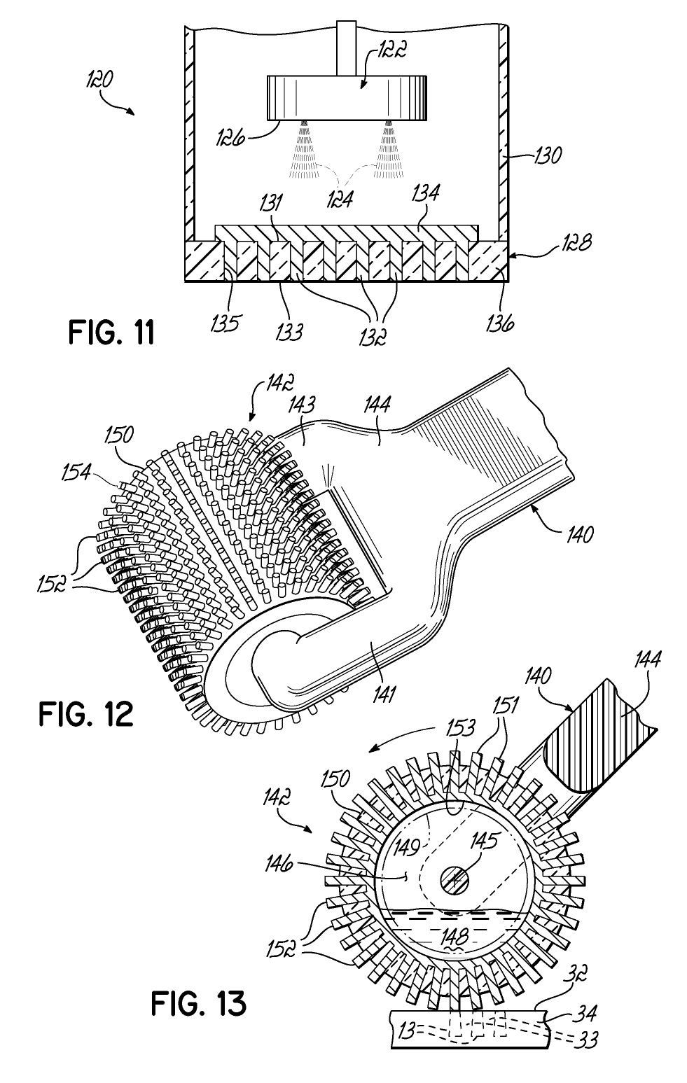

With reference to FIG. 11 and in accordance with an alternative embodiment of the invention, a delivery device 120 includes a fluid delivery member 122 configured to deliver a flow of a coolant 124 from dispensing outlets in a nozzle 126 to a coolant-receiving and a thermal-transfer member, which is generally indicated by reference numeral 128. The fluid delivery member 122 is housed inside a housing 130 to which the thermal-transfer member 128 is mechanically coupled. The coolant may be, for example, liquid nitrogen. Other commercially available refrigerants include, but are not limited to, halocarbon refrigerants such as R134a refrigerant, liquid carbon dioxide, liquid argon, liquid helium and other chemically inert and non-toxic refrigerants recognized by a person having ordinary skill in the art. Optionally, the refrigerant may be chemically inert, but not necessarily.

The thermal-transfer member 128 comprises a plurality of individual thermally-conductive energy-transfer elements 132 that are arranged in an array, which is similar to the array of electrodes 12 in FIG. 1, thermally coupled with a heat spreader 134. The energy-transfer elements 132 are embedded in passageways 135 penetrating through a thermally insulating sheet or member 136. The thermally insulating member 136 is formed from a material having a relatively low thermal conductivity, such as polyimide. In contrast, the energy-transfer elements 132 are formed from a material having a relatively high thermal conductivity, such as copper. Each energy-transfer element 132 extends between a rear side 131 that contacts the heater spreader 134 and an opposite front side 133 that contacts the skin surface 32 during a tissue treatment. The energy-transfer elements 132 may be discrete pins disposed in the passageways 135.

In an alternative embodiment, the energy-transfer elements 132 and heat spreader 134 may be an integral structure instead of an assembly of multiple discrete elements 132 with the heat spreader 134. In this regard, the energy-transfer elements 132 may be electroplated deposits formed in the passageways 135 that rely on the heat spreader 134 as a substrate. In another alternative embodiment, the heat spreader 136 may be omitted so that the rear side 131 of each energy-transfer element 132 is directly contacted or wetted by the coolant 124.

The coolant 124 operates to reduce the temperature of the thermally-conductive energy-transfer elements 132 sufficiently low to cryogenically create the microburns 13 (FIG. 5) by conductive transfer of heat from the tissue 34 (FIG. 5) to the elements 132. The arrangement and geometrical shape of the energy-transfer elements 132 may be varied to promote different treatments by altering the pattern and depth of the microburns 13. The tissue 34 underlying the skin surface 32 contacted by the insulating material of the thermally insulating member 136 between adjacent energy-transfer elements 132 is kept sufficiently warm such that damage does not occur. This promotes the formation of a pattern of microburns 13 with intervening regions 33 of healthy tissue 34.

With reference to FIGS. 12 and 13 and in accordance with an alternative embodiment of the invention, a delivery device 140 includes a chilled roller 142 that is coupled with arms 141, 143 of a forked handle 144 for rotation about an axis of rotation 145 relative to the forked handle 144. The axis of rotation 145 is generally parallel with the plane of the skin surface 32 during treatment. An interior reservoir 146 of the chilled roller 142 confines a volume of a coolant 148, which may be, for example, liquid nitrogen. The interior reservoir 146 may be lined with an optional conductive layer 149. The chilled roller 142 constitutes a coolant-receiving and thermal-transfer member that cryogenically creates microburns 13.

The chilled roller 142 comprises a cylindrical sheet or member 150 formed from a thermally insulating material and a plurality of energy-transfer members 152 that extend through the cylindrical member 150 with a sealed relationship such that the coolant 148 does not leak. Each energy-transfer member 152 extends radially relative to the axis of rotation 145 between a rear side 151 that is proximate to interior reservoir 146 and an opposite front side 153 that contacts the skin surface 32 during a tissue treatment. Each of the energy-transfer members 152 inflicts an individual microburn 13 in the tissue 34 over the contact time with the skin surface 32. The cylindrical member 150 rotates about the axis of rotation 145 in a direction generally indicated by single-headed arrow 147 when placed into contact with skin surface 32 and a forward propelling force is applied to the forked handle 144. As the cylindrical member 150 rotates, each row of energy-transfer members 152 periodically contacts the skin surface 32 for forming a corresponding row of microburns 13.

Because of the influence of the coolant 148, the energy-transfer members 152 are at a significantly lower temperature than the skin surface 32 and the tissue 34 beneath the skin surface 32. Because these regions are not in thermal equilibrium with each other, heat spontaneously flows from the region of higher temperature (i.e., the tissue 34) to the region of relatively low temperature (i.e., the energy-transfer members 152). The tissue 34 and energy-transfer members 152 exchange internal energy in an attempt to equalize the temperature of the two regions. As a result, the temperature of the tissue 34 is reduced significantly below normal body temperature and may even reach the temperature of the coolant 148, which locally damages the tissue 34 and forms the microburns 13.

The spacing between adjacent energy-transfer members 152 of each row in a direction parallel to the axis of rotation 145 determines a pitch of the microburns 13 inflicted in the tissue 34. The spacing between adjacent rows of energy-transfer members 152 likewise contributes to forming the pattern of microburns 13. The spacing between adjacent energy-transfer members 152 and adjacent rows of energy-transfer members 152 is selected such that the microburns 13 do not overlap in the tissue 34, which leaves residual healthy tissue 34 for re-growth after treatment. The density of microburns 13 is determined by the pitch and spacing between adjacent rows of energy-transfer members 152.

Although the energy-transfer members 152 are illustrated as being arranged in rows aligned generally with the axis of rotation 145, that are equally spaced about the circumference of the cylindrical member 150, the invention is not so limited. If the coolant 148 is liquid nitrogen, the temperature of the energy-transfer members 152 may be as low as liquid nitrogen temperatures (−195.79° C., 77.36 K, −320.42° F.), which is believed to more than suffice a cold enough for forming the microburns 13. However, the invention is not so limited as the microburns 13 may be formed at either higher or lower temperatures than liquid nitrogen temperatures. Freon as a coolant is an alternative exemplary type of coolant 148.

Treatment repeatability may be enhanced by endowing the chilled roller 142 with a thermal mass that is large enough to ensure that the energy-transfer members 152 are held at a constant temperature at all times. Alternatively, an active control scheme may be used in which feedback devices 154, such as temperature sensors, are placed at the distal end of one or more of the energy-transfer members 152. The angular velocity of the chilled roller 142 may be measured as the chilled roller 142 is moved across the skin surface 32 to cryogenically perform a microburn treatment. The temperature of the energy-transfer members 152 would be proportional to the angular velocity, with higher angular velocities requiring a lower temperature. This approach may be used in a control loop to produce constant-depth microburns 13 in a pattern independent of angular velocity of the chilled roller 142.

While the invention has been illustrated by a description of various embodiments and while these embodiments have been described in considerable detail, it is not the intention of the applicants to restrict or in any way limit the scope of the appended claims to such detail. Additional advantages and modifications will readily appear to those skilled in the art. Thus, the invention in its broader aspects is therefore not limited to the specific details, representative apparatus and method, and illustrative example shown and described. Accordingly, departures may be made from such details without departing from the spirit or scope of applicants' general inventive concept.

What is claimed is:

1. A device for forming a plurality of damaged regions in tissue separated by a plurality of non-damaged regions, the damaged regions and non-damaged regions located beneath a skin surface, the device comprising:
an electrode assembly positionable adjacent to the skin surface, the electrode assembly comprising a plurality of energy-delivery elements configured to deliver high frequency electrical energy to the tissue for forming the damaged regions at a corresponding plurality of locations in the tissue with adjacent locations separated by one of the non-damaged tissue regions.

2. The device of claim 1 wherein the energy-delivery elements include a plurality of first electrodes adapted to deliver the electrical energy to the tissue, the electrical energy being radiofrequency energy, and the first electrodes electrically isolated from each other.

3. The device of claim 2 wherein each of the first electrodes has a width smaller than about 300 microns, and the first electrodes have an electrode-to-electrode spacing between about 50 microns and about 4000 microns.

4. The device of claim 2 further comprising:
a radiofrequency power supply electrically coupled in an electrical circuit with the first electrodes of the electrode assembly, the radiofrequency energy power supply energizing the first electrodes to deliver the electrical energy to the tissue; and
a multiplexing network disposed in the electrical circuit, the multiplexing network adapted to open and close a current path in the electrical circuit to at least two of the first electrodes so that the first electrodes can be selectively activated.

5. The device of claim 2 wherein the electrode assembly further comprises a dielectric member having a substantially planar surface that is approximately parallel to the tissue when the damaged regions are formed, the dielectric member having a plurality of passageways aligned with the first electrodes.

6. The device of claim 5 wherein each of the first electrodes penetrates through one of the passageways in the dielectric member and projects beyond the substantially planar surface of the dielectric member and toward the tissue.

7. The device of claim 5 wherein at least one of the first electrodes is movable in a direction substantially perpendicular to the substantially planar surface of the dielectric member.

8. The device of claim 2 wherein at least one of the first electrodes includes a sidewall, a tip of a conductive material terminating the sidewall, and a dielectric shroud applied with a surrounding relationship to the sidewall such that a portion of the tip is exposed.

9. The device of claim 2 wherein at least one of the first electrodes includes a sidewall and a tip of a conductive material terminating the sidewall, the tip further including a beveled point configured to penetrate into the tissue.

10. The device of claim 2 further comprising:
a second electrode;
a first dielectric member between the second electrode and the first electrodes such that the second electrode participates in forming an electrical capacitor with each of the first electrodes and the first dielectric member when the first and second electrodes are energized; and
a second dielectric member between the first electrodes and the tissue, the second dielectric member including a plurality of openings extending in a direction from the first electrodes to the tissue, and each of the openings registered with a corresponding one of the first electrodes.

11. The device of claim 2 wherein the first electrodes have a substantially flat planar interleaved structure, and further comprising:
a second electrode including a plurality of voids, each of the first electrodes disposed in a respective one of the voids in the second electrode; and
a plurality of insulators formed from a material with higher electrical resistivity than a material forming the first and second electrodes, each of the insulators configured to electrically isolate a respective one of the first electrodes from the second electrode.

12. The device of claim 1 further comprising:
a handpiece configured to be coupled with the electrode assembly and manipulated by a clinician for positioning the electrode assembly adjacent to the skin surface.

13. The device of claim 1 wherein the energy-delivery elements comprise:
an electrode;
a dielectric member configured to be located between the electrode and the skin surface; and
a fluid containing a plurality of conductive particles configured to contact the skin surface, the conductive particles transferring the electrical energy from the electrode through the skin surface to the tissue when the electrode is energized.

14. The device of claim 1 further comprising:
an electrode;
a standoff configured to be located between the electrode and the skin surface; and
a fluid containing a plurality of conductive particles configured to contact the skin surface, the conductive particles transferring the electrical energy from the electrode through the skin surface to the tissue when the electrode is energized.

15. A device for forming a plurality of damaged regions in tissue interspaced between non-damaged regions, both located beneath a skin surface, the device comprising:
a delivery device positionable adjacent to the skin surface, the delivery device including a fluid delivery member and a plurality of thermally-conductive elements configured to contact the skin surface, the fluid delivery member configured to deliver a coolant to the thermally-conductive elements for cooling the thermally-conductive elements to a temperature sufficient to thermally form the damaged regions at a corresponding plurality of locations in the tissue with adjacent pairs of the locations separated by one of said non-damaged tissue regions.

16. The device of claim 15 wherein each of the energy-delivery elements has a front side for contacting the skin surface and a rear side opposite to the front side, and the fluid delivery member further comprises:
a sheet of an insulating material having a lower thermal conductivity than the energy-delivery elements, each of the energy-delivery elements extending through the sheet such that at least the front side is exposed; and
a nozzle having a plurality of dispensing outlets from which the coolant is delivered as a spray to the rear side of the energy-delivery elements.

17. The device of claim 16 wherein the fluid delivery member further comprises:
a heat spreader positioned between the nozzle and the energy-delivery elements for intercepting the spray of the coolant, the heat spreader thermally coupled with the energy-delivery elements for conductive heat transfer.

18. The device of claim 15 wherein each of the energy-delivery elements has a front side for contacting the skin surface and a rear side opposite to the front side, and the fluid delivery member further comprises:
a cylindrical sheet of a material having a lower thermal conductivity than the energy-delivery elements, each of the energy-delivery elements extending radially through the cylindrical sheet such that at least the front side is exposed, and the cylindrical sheet enclosing a reservoir adapted to hold the coolant; and
a handle coupled pivotally with the cylindrical sheet for circumferentially rolling the cylindrical sheet with the energy-delivery elements in contact with the skin surface.

19. A method for forming a plurality of damaged regions characteristic of a microburn pattern containing a plurality of damaged regions and a plurality of non-damaged regions in tissue beneath a skin surface, the method comprising:
transferring high frequency electrical energy between a plurality of small-area tissue contacts and the tissue; and
modifying the tissue with the electrical energy to form the damaged regions correlated with the small-area tissue contacts such that adjacent pairs of the damaged regions are separated by a respective one of the non-damaged regions.

20. The method of claim 19 wherein the high frequency electrical energy is transferred from a mutually electrically isolated plurality of first electrodes by electrical conduction to the tissue.

21. The method of claim 20 further comprising:
multiplexing the first electrodes such that current paths are sequentially opened and closed to different groups of the first electrodes.

22. The method of claim 20 wherein transferring the high frequency electrical energy further comprises:
conducting the high frequency electrical energy from the first electrodes through a conductive coupling fluid to the tissue.

23. The method of claim 20 wherein transferring the high frequency electrical energy further comprises:
contacting the first electrodes with the skin surface; and directly conducting the high frequency electrical energy from the first electrodes to the tissue.

24. The method of claim 23 wherein contacting the first electrodes with the skin surface further comprises:
retracting the first electrodes when the skin surface is initially contacted; and
advancing the first electrodes into the tissue as the damaged regions are formed.

25. The method of claim 23 wherein each of the first electrodes includes a tip, and contacting the first electrodes with the skin surface further comprises:
directly conducting the high frequency electrical energy from a portion of the tip of each of the first electrodes to the tissue.

26. The method of claim 23 wherein each of the first electrodes includes a tip and a beveled point on the tip, and contacting the first electrodes with the skin surface further comprises:
penetrating the tissue with the beveled point of each of the first electrodes.

27. The method of claim 20 wherein transferring the high frequency electrical energy further comprises:
capacitively transferring the high frequency electrical energy from a second electrode through a first dielectric material to the first electrodes.

28. The method of claim 27 wherein capacitively transferring the high frequency electrical energy further comprises:
conducting the high frequency electrical energy from the first electrodes through a plurality of openings in a second dielectric material to the tissue.

29. The method of claim 28 wherein conducting the high frequency electrical energy further comprises:
placing a conductive coupling fluid in the openings to define a plurality of individual current paths for the high frequency electrical energy from the first electrodes to the tissue.

30. The method of claim 20 wherein transferring the high frequency electrical energy further comprises:
coupling a plurality of current paths from the first electrodes through the tissue to a second electrode of a different voltage polarity in contact with the skin surface and with which the first electrodes are interleaved.

31. The method of claim 19 wherein transferring the high frequency electrical energy further comprises:
applying a plurality of electrically-conductive particles on a surface of the tissue; and
transferring the high frequency electrical energy through the electrically-conductive particles to the tissue.

32. A method for forming a plurality of damaged regions characteristic of a microburn pattern containing a plurality of damaged regions and a plurality of non-damaged regions in tissue beneath a skin surface, the method comprising:
extracting heat energy from the tissue over a plurality of small-area tissue contacts; and
cooling the tissue with the heat energy transfer at the small-area tissue contacts to an extent sufficient form the damaged regions correlated with the small-area tissue contacts such that adjacent pairs of the damaged regions are separated by a respective one of the non-damaged regions.
